(19) United States
(12) Patent Application Publication
(10) Pub. No.: US 2010/0145125 A1
(43) Pub. Date: Jun. 10, 2010

(54) PROCESS OF PRODUCING LIGHT OLEFINS THROUGH THE CONVERSION OF METHANOL AND ETHANOL

(75) Inventors: Zaiku Xie, Shanghai (CN); Guozhen Qi, Shanghai (CN); Huiming Zhang, Shanghai (CN); Siqing Zhong, Shanghai (CN)

Correspondence Address: SCULLY SCOTT MURPHY & PRESSER, PC, 400 GARDEN CITY PLAZA, SUITE 300, GARDEN CITY, NY, 11530, US

(73) Assignees: CHINA PETROLEUM & CHEMICAL CORPORATION, Beijing (CN); Shanghai Research Institute of Petrochemical Technology Sinopec, Shanghai (CN)

(21) Appl. No.: 12/526,408

(22) PCT Filed: Feb. 4, 2008
(86) PCT No.: PCT/CN08/00310

§ 371 Date: Nov. 12, 2009

(30) Foreign Application Priority Data
Feb. 7, 2007 (CN) 200710037233.9

(51) Int. Cl. C07C 1/20

(52) U.S. Cl. 585/639

(57) ABSTRACT

The present invention discloses a process of producing light olefins through the conversion of methanol and ethanol. The process comprises: feeding a first portion of a feed via a distributor at the bottom of a fluidized-bed reactor to a reaction zone containing a catalyst; feeding a second portion of the feed from at least one location above the distributor to the reaction zone; contacting the feed with the catalyst and allowing it to react, to give a stream containing ethylene and propylene; and withdrawing the stream containing ethylene and propylene from the top of the reactor, and passing it to a separation system to separate ethylene and propylene, wherein the first portion of the feed and the second portion of the feed comprises each independently methanol or ethanol or the both, with a proviso that the total feed comprises both methanol and ethanol, and a weight ratio of methanol to ethanol in the total feed is in a range of from 99:1 to 0.1:1.

### CROSS REFERENCE OF RELATED APPLICATIONS

The present application claims the benefit of the Chinese Patent Application No. 200710037233.9, filed on Feb. 7, 2007, which is incorporated herein by reference in its entirety and for all purposes.

### FIELD OF THE INVENTION

The present invention relates to a process of producing light olefins through the conversion of methanol and ethanol.

### BACKGROUND OF THE INVENTION

Light olefins, defined as ethylene and propylene in the present invention, are important basic chemical feedstock, and the demand for them is increasing. At present, ethylene and propylene are mainly produced from petroleum feedstock by catalytic cracking or steam cracking. However, as petroleum resources are being exhausted and their prices are rising increasingly, other approaches for producing ethylene and propylene are paid more and more attention.

An important approach for producing light olefins from non-petroleum feedstock is the conversion of oxygenates, for example, lower alcohols (methanol, ethanol), ethers (dimethyl ether, methyl ethyl ether), esters (dimethyl carbonate, methyl formate) and the like to olefins, especially the conversion of lower alcohols to light olefins. The production of light olefins from methanol is a promising process, because methanol can be produced in large scale from coal or natural gas via syngas. However, the methanol-to-olefin (MTO) process suffers from the lower selectivity to light olefins, and it requires complicated heat management because the reaction is a strongly exothermal reaction.

A possible approach for enhancing the selectivity to light olefins in MTO process is adding a diluent and thereby conducting the conversion at a lower feed partial pressure, which favors thermodynamically the formation of olefins. However, as the amount of the diluent added increases, additional costs for producing the diluent and equipments used to condense and recover the diluent are required, and the addition of the diluent will greatly increase the size of the equipment so that the production costs will be greatly increased.

A process with staged injection of feed has also been suggested to use in MTO process to enhance selectivity to light olefins. For example, CN1190395C applies the technique of staged injection of feed to a fluidized-bed reactor used to convert oxygenate to olefins, wherein methanol or dimethyl ether is introduced to a reaction zone at multiple injection locations along the flow axis of the fluidized-bed reactor.

On the other hand, ethanol-to-ethylene (ETO) process is known, and the process has a higher selectivity to ethylene. Furthermore, a lower partial pressure of the feed also favors the enhancement of the selectivity to ethylene. At present, ETO process suffers from problems such as small production scale of the feed and poor process economics.

No process integrating MTO process and ETO process has been disclosed in the prior art. It is well known that reaction temperature in ETO process is generally lower than 400° C., while reaction temperature in MTO process is generally from 450° C. to 500° C., in order to maintain a highest total selectivity to ethylene plus propylene. Thus, the difference in process condition is one of obstacles for integrating MTO process and ETO process. Furthermore, the catalyst used in conventional ETO process is non-molecular sieve catalyst, such as alumina or the like. There are little reports on the successfully carrying out of ETO process by using a zeolite molecular sieve catalyst or a non-zeolite molecular sieve catalyst. This is another obstacle for integrating said two processes.

The present invention converts methanol and ethanol to light olefins using a molecular sieve catalyst in the same reactor under the same process conditions, and solve the problems suffered by the prior art.

### SUMMARY OF THE INVENTION

The inventors have found that, by integrating MTO process and ETO process, i.e., using methanol and ethanol in combination to produce light olefins, the problem of lower selectivity to light olefins for MTO process and the problem of poor economics for ETO process are solved, and reaction heat management is rendered easier. By staged injection of feed, it is possible to further improve the selectivity to light olefins and reaction heat management. The process of the invention is especially suitable for developing ethylene and propylene industry at a location where a large amount of methanol and a minor amount of ethanol are available.

An object of the invention is to provide a process for converting methanol and ethanol to light olefins, comprising:

feeding a first portion of a feed via a distributor at the bottom of a fluidized-bed reactor to a reaction zone containing a catalyst;

feeding a second portion of the feed from at least one location above the distributor to the reaction zone;

contacting the feed with the catalyst and allowing it to react, to give a stream containing ethylene and propylene; and

withdrawing the stream containing ethylene and propylene from the top of the reactor, and passing it to a separation system to separate ethylene and propylene,

wherein the first portion of the feed and the second portion of the feed comprises each independently methanol or ethanol or the both, with a proviso that the total feed comprises both methanol and ethanol, and a weight ratio of methanol to ethanol in the total feed is in a range of from 99:1 to 0.1:1.

### BRIEF DESCRIPTION OF THE DRAWINGS

The above object as well as other objects of the present invention will be apparent from the following detailed description on the present invention with reference to the drawings, wherein

FIG. 1 is a schematic of an embodiment of the reactors useful in the process of the present invention.

### DETAILED DESCRIPTION OF PREFERRED EMBODIMENTS

The present invention provides a process for converting methanol and ethanol to light olefins, comprising: feeding a first portion of a feed via a distributor at the bottom of a fluidized-bed reactor to a reaction zone containing a catalyst; feeding a second portion of the feed from at least one location above the distributor to the reaction zone; contacting the feed with the catalyst and allowing it to react, to give a stream containing ethylene and propylene; and withdrawing the stream containing ethylene and propylene from the top of the reactor, and passing it to a separation system to separate ethylene and propylene, wherein the first portion of the feed and the second portion of the feed comprises each independently methanol or ethanol or the both, with a proviso that the total feed comprises both methanol and ethanol, and a weight ratio of methanol to ethanol in the total feed is in a range of from 99:1 to 0.1:1.

In the process of the invention, a combination of methanol and ethanol is used as feed to produce light olefins. Since the conversion of methanol to olefin is a highly exothermal reaction and the conversion of ethanol to olefin is a highly endothermal reaction, the use of methanol and ethanol in combination as feed will significantly favor the management of reaction heat. And, without being limited to any theory, it is believed that methanol and ethanol function as a diluent for each other, thereby favoring the enhancement of selectivity to the light olefin product. In order to favor the enhancement of the selectivity to the light olefin product and to improve heat management, the weight ratio of methanol to ethanol in the total feed is from 99:1 to 0.1:1, preferably from 50:1 to 2:1.

In an embodiment of the present invention, the weight ratio of the first portion of feed to the second portion of feed is in a range of from 1:3 to 20:1, preferably from 1:2 to 15:1, more preferably from 1:1.5 to 10:1, and most preferably from 1:1 to 8:1. The first portion of feed and the second portion of feed may have the same or different composition. However, conveniently, the first portion of feed and the second portion of feed have the same composition.

In an embodiment of the present invention, the second portion of feed is fed to the reaction zone from horizontally and/or vertically spaced multiple locations above the distributor. Feeding the second portion of feed from horizontally spaced multiple locations will favor a uniform distribution of the feed in the reaction zone. Feeding the second portion of feed from vertically spaced multiple locations will favor the enhancement of selectivity to light olefins. In this embodiment, the streams of the second portion of feed fed from the multiple locations may have the same or different composition. However, conveniently, especially in a case where horizontally spaced multiple injection ports are utilized, the streams of the second portion of feed fed from the multiple injection ports have the same composition. In a preferred embodiment, the second portion of feed is fed to the reaction zone from vertically spaced multiple locations above the distributor.

The location of the injection port(s) for the second portion of feed may vary in a broad range along the axis direction of the reactor, but in generally in a range of from 1/10 to ⅘, preferably from ⅕ to ⅗, and more preferably from ⅕ to ½ reaction zone height above the distributor at the reactor bottom. If multiple injection ports spaced along the axis direction of the reactor are employed, the number of the injection ports may vary broadly. However, overmuch injection ports not only increase complicacy of the equipment but also inconvenience the maintenance, even affect the flow behavior of reagents in the reaction zone. In addition, when the number of the injection ports spaced along the axis direction of the reactor increases to a certain level or the location of an injection port is too high, the conversion of the feed may decrease to an unacceptable level, while the increment in selectivity to light olefins decreases. Thus, the number and location of the injection ports should be suitably set under the precondition that the conversion of the feed is acceptable. The amount of reagents fed from individual injection ports may be the same or different.

Optionally, any portion of the feed in the process of the invention may comprise a diluent known by those skilled in the art. The diluent may be at least one selected from the group consisting of C₁ to C₄ alkane, for example methane, ethane, propane, n-butane and iso-butane; C₃ to C₄ alcohol, for example n-propanol, iso-propanol, n-butanol and iso-butanol; CO; CO₂; nitrogen; steam; and monocyclic arene, for example benzene and toluene. Preferably, the diluent is at least one selected from the group consisting of C₁ to C₄ alkanes, C₃ to C₄ alcohols and steam, and more preferably steam.

In an embodiment of the present invention, the fluidized-bed reactor is a vertical fluidized-bed reactor, preferably a dense phase fluidized-bed reactor or a fast fluidized-bed reactor, and more preferably a dense phase fluidized-bed reactor.

In an embodiment of the present invention, the present process may employ the following process conditions: a temperature inside the reaction zone in the fluidized-bed reactor ranging from 350° C. to 450° C., and preferably from 375° C. to 425° C.; a weight hourly space velocity (WHSV) of the feed ranging from 0.5 to 50 h⁻¹, and preferably from 1 to 20 h⁻¹; and a reaction pressure ranging from 0 to 1 MPa (gauge), and preferably from 0 to 0.3 MPa (gauge).

It is known by those skilled in the art that the reaction temperature commonly used in ETO reaction is lower than that commonly used in MTO reaction, because over high reaction temperature may result in the increase of selectivity to acetaldehyde in the ETO reaction. Furthermore, over high reaction temperature may increase the probability of decomposition of methanol or ethanol into inorganic carbon (such as COₓ). Therefore, in order to obtain a higher selectivity to light olefins and to enhance utilization of carbon in the feed (i.e., generating less alkanes and inorganic carbon), the selection of reaction temperature is important. As well known by those skilled in the art, it is possible to adjust proportion of ethylene and propylene in the MTO reaction product by adjusting reaction temperature at a lower reaction temperature, selectivity to propylene increases so that the ratio of propylene/ethylene (P/E) increases. When the reaction is carried out in the reaction temperature range described above, MTO reaction product is predominately propylene, while the ETO reaction product is mostly ethylene. Reaction temperature and ratio of methanol to ethanol in the total feed may be suitably chosen depending on the desired P/E ratio.

The catalyst useful in the process of the invention may be any of molecular sieve catalysts known by those skilled in the art, as long as it is suitable for MTO process and ETO process. In a preferred embodiment of the present invention, the catalyst comprises one or more ZSM or SAPO molecular sieves, more preferably ZSM-5 and/or SAPO-34 molecular sieve, and most preferably SAPO-34 molecular sieve. The catalyst comprises optionally a matrix known by those skilled in the art, such as silica, alumina, titania, zirconia, magnesia, thoria, silica-alumina, various clays, and mixtures thereof. The techniques to prepare suitable molecular sieve catalyst are known by those skilled in the art.

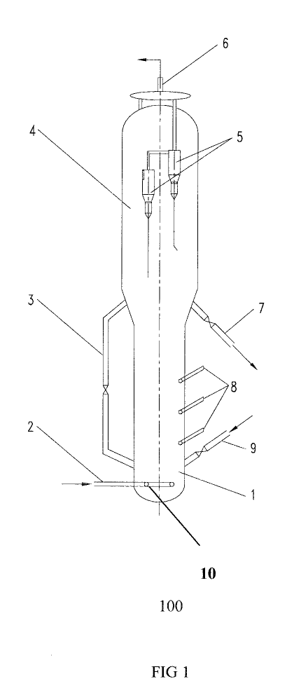

With reference to FIG. 1, an embodiment of the present invention will be described below, wherein the reactor is a dense phase fluidized-bed reactor. However, as indicated above, the process of the invention may also employ, for example, a fast fluidized-bed reactor. As shown in FIG. 1, a first portion of feed is fed from the bottom of reactor 100 via line 2 and distributor 10 into reaction zone 1 containing catalyst. The distributor 10 may be in the form of nozzle, porous distribution plate, tube distributor, or the like. The first portion of feed is fed at least partially in gas state into the reaction zone 1, to maintain the catalyst in the reaction zone 1 in fluidizing state. A second portion of feed is fed into the reaction zone 1 via one or more injection ports 8 (in this case, three) spaced along the axis of the reactor. The first portion of feed and/or the second portion of feed may be heat exchanged with the catalyst carrying an amount of heat (not shown), and enter(s) the reaction zone 1 after having been heated to a desired temperature. The catalyst carrying an amount of heat may be the one in the transporting line from the reactor 100 to a regenerator (not shown) or from the regenerator to the reactor 100.

The first portion of feed and the second portion of feed contact with the catalyst in the reaction zone 1 and react, to form a product stream containing ethylene and propylene. The product stream entraining some of catalyst enters upwards a gas-solid separation zone 4, where it is separated by a cyclone 5 located therein into a gaseous product stream and a solid catalyst stream. The gaseous product stream enters subsequent separation stage via outlet line 6, to isolate ethylene and propylene by a process well known by those skilled in the art. The solid catalyst is collected in the lower portion of the separation zone 4. The solid catalyst in the lower portion of the separation zone 4 may be circulated to the reaction zone 1 via a catalyst circulation line 3 or sent to the regenerator via a line 7 to be regenerated. The regenerated catalyst is returned to the reaction zone 1 via a line 9. It is possible to adjust the amount of the catalyst circulated into the reaction zone 1 via the catalyst circulation line 3 and the amount of the catalyst returned to the reaction zone 1 from the regenerator via the line 9, and/or the regeneration extent of the catalyst, so as to suitably adjust the average amount of coke on the catalyst in the reaction zone 1, thereby to adjust the selectivity of reaction in the reaction zone. Catalyst regeneration processes are known by those skilled in the art, for example one by burning off coke in an oxygen-containing atmosphere. Prior to the regeneration, the coked catalyst withdrawn from the reactor is optionally stripped, to recover volatile carbonaceous material adsorbed thereon.

The present process for producing light olefins renders the heat management easy, and achieves a higher yield of ethylene and propylene, for example up to 52.7 wt %.

### EXAMPLES

The following examples are given for further illustrating the invention, but do not make limitation to the invention in any way.

In the following Examples, methanol conversion and ethanol conversion means:

% methanol conversion=(inlet methanol mass flow rate−outlet methanol mass flow rate)/inlet methanol mass flow rate×100, and

% ethanol conversion=(inlet ethanol mass flow rate−outlet ethanol mass flow rate)/inlet ethanol mass flow rate×100.

In the following Examples, ethylene yield and propylene yield means:

% ethylene yield=(outlet ethylene mass flow rate/inlet total mass flow rate of methanol and ethanol)×100, and

% propylene yield=(outlet propylene mass flow rate/inlet total mass flow rate of methanol and ethanol)×100.

### Example 1

In a mini dense phase fluidized-bed reactor, an experiment was carried out by using a SAPO-34 molecular sieve catalyst molded by spray drying comprising 50 wt % of SAPO-34 molecular sieve and 50 wt % of alumina matrix. A 99:1 by weight mixture of methanol and ethanol was used as feed. The feed was split into a first portion of feed and a second portion of feed in 8:1 weight ratio, and they were fed to a reaction zone via a distributor at the bottom of the reactor and one injection port on the wall of the reactor, respectively. The injection port was ⅓ reaction zone height away from the bottom distributor. Reaction temperature was 375° C., WHSV of the feed was 1.0 h⁻¹, and reaction pressure was 0 MPa (gauge). Reaction product was analyzed by an in-line gas chromatogragh. The results obtained when the experiment had been run for 10 min are as follows: methanol conversion is 96.6 wt %, ethanol conversion is 100 wt %, ethylene yield is 18.4 wt %, and propylene yield is 13.2 wt %.

### Example 2

An experiment was carried out according to the procedure as described in Example 1, except that reaction temperature was changed to 425° C. The results obtained when the experiment had been run for 10 min are as follows: methanol conversion is 98.7 wt %, ethanol conversion is 100 wt %, ethylene yield is 21.3 wt %, and propylene yield is 11.4 wt %.

### Example 3

An experiment was carried out according to the procedure as described in Example 1, except that reaction temperature was changed to 350° C., and weight ratio of methanol to ethanol in the feed was 0.1:1. The results obtained when the experiment had been run for 10 min are as follows: methanol conversion is 100 wt %, ethanol conversion is 97.2 wt %, ethylene yield is 44.2 wt %, and propylene yield is 3.9 wt %.

### Example 4

An experiment was carried out according to the procedure as described in Example 3, except that the weight ratio of the first portion of feed to the second portion of feed was changed to 1:1. The results obtained when the experiment had been run for 10 min are as follows: methanol conversion is 100 wt %, ethanol conversion is 95.1 wt %, ethylene yield is 46.7 wt %, and propylene yield is 4.4 wt %.

### Example 5

An experiment was carried out according to the procedure as described in Example 1, except that the reaction temperature was changed to 450° C., and the injection port was ½ reaction zone height away from the bottom distributor. The results obtained when the experiment had been run for 10 min are as follows: methanol conversion is 98.7 wt %, ethanol conversion is 100 wt %, ethylene yield is 20.3 wt %, yield of propylene is 13.8 wt %.

### Example 6

An experiment was carried out according to the procedure as described in Example 5, except that a fast fluidized bed reactor was used, and WHSV of the feed was 20 h⁻¹. The results obtained when the experiment had been run for 10 min are as follows: methanol conversion is 94.9 wt %, ethanol conversion is 99.7 wt %, ethylene yield is 21.4 wt %, and propylene yield is 10.9 wt %.

### Example 7

An experiment was carried out according to the procedure as described in Example 6, except that the reaction pressure (gauge) was changed to 1 MPa, and WHSV of the feed was 50 h⁻¹. The results obtained when the experiment had been run for 10 min are as follows: methanol conversion is 90.4 wt %, ethanol conversion is 98.6 wt %, ethylene yield is 15.7 wt %, and propylene yield is 10.2 wt %.

### Example 8

An experiment was carried out according to the procedure as described in Example 1, except that the reaction pressure (gauge) was changed to 0.3 MPa, and WHSV of the feed is 0.5 h⁻¹. The results obtained when the experiment had been run for 10 min are as follows: methanol conversion is 98.1 wt %, ethanol conversion is 100 wt %, ethylene yield is 16.5 wt %, and propylene yield is 11.8 wt %.

### Example 9

Experiments were carried out according to the procedure as described in Example 1, except that ZSM-34, ZSM-5, SAPO-18, and SAPO-17 molecular sieve catalysts were separately used as the catalyst. The results obtained when the experiments had been run for 10 min are shown in Table 1 below.

TABLE 1

| | Catalyst | | | |
|---|---|---|---|---|
| | ZSM-34* | ZSM-5* | SAPO-18* | SAPO-17* |
| Methanol Conversion, wt % | 93.5 | 98.8 | 96.0 | 95.4 |
| Ethanol Conversion, wt % | 96.1 | 100 | 100 | 90.1 |
| Ethylene Yield, wt % | 8.4 | 5.8 | 17.7 | 6.4 |
| Propylene Yield, wt % | 10.1 | 14.7 | 12.2 | 9.7 |

*comprising 50 wt % of the indicated molecular sieve and 50 wt % of alumina and prepared by spray drying.

### Example 10

An experiment was carried out according to the procedure as described in Example 1, except that the second portion of feed was split into two streams in 1:1 weight ratio, and the two streams were fed through two injection ports located along the axis of the reaction zone at ⅓ reaction zone height and ½ reaction zone height away from the bottom distributor, respectively. The results obtained when the experiment had been run for 10 min are as follows: methanol conversion is 95.0 wt %, ethanol conversion is 100 wt %, ethylene yield is 19.6 wt %, and propylene yield is 13.8 wt %.

### Example 11

An experiment was carried out according to the procedure as described in Example 1, except that the weight ratio of methanol to ethanol in the feed was changed to 1:1, and methanol was fed into the reaction zone from the distributor at the reactor bottom, and ethanol was fed into the reaction zone from an injection port on the wall of the reactor, which injection port was ⅓ reaction zone height away from the bottom distributor. The results obtained when the experiment had been run for 10 min are as follows: methanol conversion is 95.9 wt %, ethanol conversion is 99.6 wt %, ethylene yield is 35.2 wt %, and propylene yield is 10.9 wt %.

### Example 12

An experiment was carried out according to the procedure as described in Example 1, except that the weight ratio of methanol to ethanol in the feed was changed to 1:1, and ethanol was fed into the reaction zone from the distributor at the reactor bottom, and methanol was fed into the reaction zone through four injection ports spaced vertically on the wall of the reactor. The four injection ports were ⅛ reaction zone height, ⅙ reaction zone height, ¼ reaction zone height, and ½ reaction zone height away from the bottom distributor, respectively. The results obtained when the experiment had been run for 10 min are as follows: methanol conversion is 94.7 wt %, ethanol conversion is 100 wt %, ethylene yield is 36.3 wt %, and propylene yield is 9.7 wt %.

### Example 13

An experiment was carried out according to the procedure as described in Example 1, except that the weight ratio of methanol to ethanol in the feed was changed to 1:1, wherein 50 wt % of ethanol and the total methanol as a first portion of feed were fed into the reaction zone from the distributor at the bottom of the reactor, the remaining 50 wt % of ethanol as a second portion of feed was fed into the reaction zone from an injection port on the wall of the reactor, which was ⅓ reaction zone height away from the bottom distributor, and the weight ratio of the first portion of feed to the second portion of feed was 3:1. The results obtained when the experiment had been run for 10 min are as follows: methanol conversion is 98.4 wt %, ethanol conversion is 98.8 wt %, ethylene yield is 37.2 wt %, and propylene yield is 9.6 wt %.

### Example 14

An experiment was carried out according to the procedure as described in Example 1, except that the weight ratio of methanol to ethanol in the feed was changed to 1:1, wherein 50 wt % of methanol and the total ethanol as a first portion of feed were fed into the reaction zone from the distributor at the bottom of the reactor, the remaining 50 wt % of methanol as a second portion of feed was fed into the reaction zone from an injection port on the wall of the reactor, which was ⅓ reaction zone height away from the bottom distributor, and the weight ratio of the first portion of feed to the second portion of feed was 3:1. The results obtained when the experiment had been run for 10 min are as follows: methanol conversion is 93.9 wt %, ethanol conversion is 99.5 wt %, ethylene yield is 36.8 wt %, and propylene yield is 10.2 wt %.

### Example 15

An experiment was carried out according to the procedure as described in Example 1, except that the weight ratio of methanol to ethanol in the feed was changed to 2:1, the reaction temperature was changed to 400° C., and the weight ratio of the first portion of feed to the second portion of feed was changed to 4:1. The results obtained when the experiment had been run for 10 min are as follows: methanol conversion is 98.6 wt %, ethanol conversion is 100 wt %, ethylene yield is 42.4 wt %, and propylene yield is 10.3 wt %.

### Example 16

An experiment was carried out according to the procedure as described in Example 1, except that the weight ratio of methanol to ethanol in the feed was changed to 50:1, the WHSV of the feed was 10.0 h⁻¹, and reaction pressure was 0.1 MPa (gauge). The results obtained when the experiment had been run for 10 min are as follows: methanol conversion is 94.9 wt %, ethanol conversion is 99.6 wt %, ethylene yield is 16.8 wt %, and propylene yield is 13.9 wt %.

### Example 17

An experiment was carried out according to the procedure as described in Example 1, except that the weight ratio of methanol to ethanol in the feed was changed to 10:1. The results obtained when the experiment had been run for 10 min are as follows: methanol conversion is 96.8 wt %, ethanol conversion is 100 wt %, ethylene yield is 19.1 wt %, and propylene yield is 13.1 wt %.

### Example 18

An experiment was carried out according to the procedure as described in Example 1, except that the weight ratio of methanol to ethanol in the feed was changed to 7:1, wherein 80 wt % of methanol as a first portion of the feed was fed to the reaction zone from the distributor at the bottom of the reactor, the remaining 20 wt % of methanol and total ethanol as a second portion of the feed were fed to the reaction zone from an injection port on the wall of the reactor, which was ⅓ reaction zone height away from the bottom distributor, and weight ratio of the first portion of feed to the second portion of feed was 2.3:1. The results obtained when the experiment had been run for 10 min are as follows: methanol conversion was 97.1 wt %, conversion of ethanol was 99.8 wt %, yield of ethylene was 21.2 wt %, and yield of propylene was 12.4 wt %.

### Example 19

An experiment was carried out according to the procedure as described in Example 1, except that the weight ratio of methanol to ethanol in the feed was changed to 7:1, wherein the total ethanol as a first portion of the feed was fed into the reaction zone from the distributor at the bottom of the reactor, and the total methanol as a second portion of the feed was fed into the reaction zone from an injection port on the wall of the reactor, which was ⅓ reaction zone height away from the distributor at the bottom. The results obtained when the experiment had been run for 10 min are as follows: methanol conversion is 93.2 wt %, ethanol conversion is 100 wt %, ethylene yield is 30.5 wt %, and propylene yield is 11.3 wt %.

### Example 20

An experiment was carried out according to the procedure as described in Example 1, except that the weight ratio of methanol to ethanol in the feed was changed to 0.5:1, wherein 80 wt % of ethanol as a first portion of feed was fed into the reaction zone from the distributor at the bottom of the reactor, the remaining 20 wt % of ethanol and the total methanol as a second portion of feed were fed into the reaction zone from an injection port on the wall of the reactor, which was ⅓ reaction zone height away from the distributor at the bottom, and the weight ratio of the first portion of feed to the second portion of feed was 1.14:1. The results obtained when the experiment had been run for 10 min are as follows: methanol conversion is 99.2 wt %, ethanol conversion is 99.4 wt %, ethylene yield is 38.0 wt %, and propylene yield is 9.2 wt %.

### Example 21

An experiment was carried out according to the procedure as described in Example 1, except that the weight ratio of methanol to ethanol in the feed was changed to 26:1, and the weight ratio of the first portion of feed to the second portion of feed was changed to 5:1. The results obtained when the experiment had been run for 10 min are as follows: methanol conversion is 97.2 wt %, ethanol conversion is 100 wt %, ethylene yield is 20.4 wt %, and propylene yield is 12.7 wt %.

### Example 22

An experiment was carried out according to the procedure as described in Example 1, except that the weight ratio of methanol to ethanol in the feed was changed to 38:1, and the weight ratio of the first portion of feed to the second portion of feed was changed to 7:1. The results obtained when the experiment had been run for 10 min are as follows: methanol conversion is 97.0 wt %, ethanol conversion is 100 wt %, ethylene yield is 19.9 wt %, and propylene yield is 12.9 wt %.

### Example 23

An experiment was carried out according to the procedure as described in Example 1, except that the weight ratio of methanol to ethanol in the feed was changed to 66:1, and the weight ratio of the first portion of feed to the second portion of feed was changed to 5:1. The results obtained when the experiment had been run for 10 min are as follows: methanol conversion is 96.8 wt %, ethanol conversion is 100 wt %, ethylene yield is 19.1 wt %, and propylene yield is 13.1 wt %.

### Example 24

An experiment was carried out according to the procedure as described in Example 1, except that the weight ratio of methanol to ethanol in the feed was changed to 85:1, and the weight ratio of the first portion of feed to the second portion of feed was changed to 5:1. The results obtained when the experiment had been run for 10 min are as follows: methanol conversion is 96.7 wt %, ethanol conversion is 100 wt %, ethylene yield is 18.7 wt %, and propylene yield is 13.7 wt %.

### Comparative Example 1

An experiment was carried out by using the reaction apparatus and catalyst as described in Example 1 according to the procedure as described in Example 1, except that the feed was changed to methanol, and the weight ratio of the first portion of feed to the second portion of feed was 1:1. The results obtained when the experiment had been run for 10 min are as follows: methanol conversion is 94.2 wt %, ethylene yield is 14.8 wt %, and propylene yield is 14.9 wt %.

### Comparative Example 2

An experiment was carried out by using the reaction apparatus and catalyst as described in Example 1 according to the procedure as described in Example 1, except that the feed was changed to ethanol, and the weight ratio of the first portion of feed to the second portion of feed was 1:1. The results obtained when the experiment had been run for 10 min are as follows: conversion of ethanol is 98.3 wt %, ethylene yield is 44.1 wt %, and propylene yield is 2.2 wt %.

### Comparative Example 3

An experiment was carried out by using the reaction apparatus and catalyst as described in Example 1 according to the procedure as described in Example 1, except that the feed was changed to methanol, and all the feed was fed into the reaction zone from the distribution plate at the bottom of the reactor. The results obtained when the experiment had been run for 10 min are as follows: methanol conversion is 95.8 wt %, ethylene yield is 13.4 wt %, and propylene yield is 13.7 wt %.

While the invention has been described with reference to exemplary embodiments, it will be understood by those skilled in the art that various changes and modifications may be made without departing from the spirit and scope of the invention. Therefore, the invention is not limited to the particular embodiments disclosed as the best mode contemplated for carrying out this invention, but the invention will include all embodiments falling within the scope of the appended claims.

1. A process for converting methanol and ethanol to light olefins, comprising:
feeding a first portion of a feed via a distributor at the bottom of a fluidized-bed reactor to a reaction zone containing a catalyst;
feeding a second portion of the feed from at least one location above the distributor to the reaction zone;
contacting the feed with the catalyst and allowing it to react, to give a stream containing ethylene and propylene; and
withdrawing the stream containing ethylene and propylene from the top of the reactor, and passing it to a separation system to separate ethylene and propylene,
wherein the first portion of the feed and the second portion of the feed comprises each independently methanol or ethanol or the both, with a proviso that the total feed comprises both methanol and ethanol, and a weight ratio of methanol to ethanol in the total feed is in a range of from 99:1 to 0.1:1.

2. The process of claim 1, wherein the weight ratio of methanol to ethanol in the total feed is in a range of from 50:1 to 2:1.

3. The process of claim 1, wherein a weight ratio of the first portion of the feed to the second portion of the feed is in a range of from 1:3 to 20:1.

4. The process of claim 3, wherein the weight ratio of the first portion of the feed to the second portion of the feed is in a range of from 1:1 to 8:1.

5. The process of claim 1, wherein the fluidized-bed reactor is a dense phase fluidized-bed reactor or a fast fluidized-bed reactor.

6. The process of claim 1, wherein the fluidized-bed reactor is a dense phase fluidized-bed reactor.

7. The process of claim 1, wherein the process is carried out under the following conditions: a temperature inside the reaction zone in the fluidized-bed reactor ranging from 350° C. to 450° C., a total WHSV of the feed ranging from 0.5 to 50 h⁻¹, and a reaction pressure ranging from 0 to 1 MPa (gauge), and wherein the catalyst comprises one or more ZSM molecular sieves or SAPO molecular sieves.

8. The process of claim 1, wherein the process is carried out under the following conditions: a temperature inside the reaction zone in the fluidized-bed reactor ranging from 375° C. to 425° C., a total WHSV of the feed ranging from 1 to 20 h⁻¹, and a reaction pressure ranging from 0 to 0.3 MPa (gauge), and wherein the catalyst comprises ZSM-5 molecular sieve and/or SAPO-34 molecular sieve.

9. The process of claim 7, wherein the catalyst comprises SAPO-34 molecular sieve.

10. The process of claim 7, wherein the catalyst further comprises one or more matrices.

11. The process of claim 1, wherein the first portion of the feed and/or the second portion of the feed further comprise(s) at least one diluent selected from the group consisting of C₁ to C₄ alkane, C₃ to C₄ alcohol, CO, CO₂, nitrogen, steam, and monocyclic arene.
